(19) United States
(12) Patent Application Publication
(10) Pub. No.: US 2023/0241379 A1
(43) Pub. Date: Aug. 3, 2023

(54) Entropy reduction instrument

(71) Applicant: Sichuan Zhonggu Jiuhe Medical Equipment Co., Ltd., Chengdu (CN)

(72) Inventors: Cunzheng SHEN, Chengdu (CN); Xiaobin SHEN, Chengdu (CN); Xiaowen SHEN, Chengdu (CN)

(21) Appl. No.: 18/129,110

(22) Filed: Mar. 31, 2023

(30) Foreign Application Priority Data
May 30, 2022 (CN) 202210597969.6

(51) Int. Cl. A61N 1/20; A61N 1/04

(52) U.S. Cl. CPC A61N 1/205; A61N 1/0476

(57) ABSTRACT

An entropy reduction instrument, including a housing, a battery assembly, a negentropy separator, an electrode plate assembly, and a power supply interface. The housing is provided with an accommodating cavity inside. The battery assembly is configured to generate electrical energy containing negentropic energy. The negentropy separator is fixedly arranged in the accommodating cavity, and is configured to receive the electrical energy output from the battery assembly and separate the negentropic energy from the electrical energy. The electrode plate assembly is connected to the negentropy separator, and is configured for receiving and processing the negentropic energy to generate uniform negentropic emission energy and apply it to the human body. The power supply interface is connected to the battery assembly and the negentropy separator, and is configured to supply power to the battery assembly, the negentropy separator, and the electrode plate assembly after being connected to an external power supply.

### CROSS-REFERENCE TO RELATED APPLICATIONS

This application claims the benefit of priority from Chinese Patent Application No. 202210597969.6, filed on May 30, 2022. The content of the aforementioned application, including any intervening amendments thereto, is incorporated herein by reference in its entirety.

### TECHNICAL FIELD

This application relates to rehabilitation instruments, and more particularly to an entropy reduction instrument.

### BACKGROUND

With the development of society and science technology the improvement of people's living standards, more and more attention has been paid to the health maintaining and life prolonging. In daily life, people can enhance their physical fitness through physical exercise or some medical equipment. Although these means have a certain life prolonging effect, they still cannot essentially play a role in delaying aging, maintaining health and prolonging the life.

### SUMMARY

An object of the present disclosure is to provide an entropy reduction instrument to overcome at least one of the above-mentioned technical problems. The entropy reduction instrument includes a housing, a battery assembly, a negentropy separator, and an electrode plate assembly. The entropy reduction instrument provided herein can reorganize the human micro-structure at the particle level to reach the original healthy state. Moreover, it also plays a role in activating pare cells, repairing the damaged cells, and reducing the entropy to reach an ordered state, thereby delaying the aging, and prolonging the human life.

Technical solutions of the present disclosure are described as follows.

The present disclosure provides an entropy reduction instrument, comprising:

a housing;

a battery assembly;

a negentropy separator;

an electrode plate assembly; and

a power supply interface;

wherein the housing is provided with an accommodating cavity inside;

the battery assembly is configured to generate electrical energy; and the electrical energy contains negentropic energy;

the negentropy separator is fixedly arranged in the accommodating cavity, and connected to the battery assembly; and the negentropy separator is configured to receive the electrical energy output from the battery assembly and separate the negentropic energy from the electrical energy;

the electrode plate assembly is connected to the negentropy separator, and is configured for receiving and processing the negentropic energy separated by the negentropy separator to generate uniform negentropic emission energy and apply the uniform negentropic emission energy to human body; and

the power supply interface is connected to the battery assembly and the negentropy separator; and the power supply interface is configured to supply power to the battery assembly, the negentropy separator, and the electrode plate assembly after being connected to an external power supply.

In some embodiments, the entropy reduction instrument further comprises a negentropy digital voltmeter, an oscilloscope, and a display;

wherein the negentropy digital voltmeter is connected to the negentropy separator, and is configured to detect an output voltage of the negentropy separator;

the oscilloscope is connected to the negentropy separator, and is configured for monitoring physical parameters and waveform state of the negentropy separator; and

the display is connected to the oscilloscope, and is configured to synchronously transmit a display interface of the oscilloscope.

In some embodiments, the entropy reduction instrument further comprises a negentropy switch, a power switch, and a power indicator;

wherein the negentropy switch is connected to the negentropy separator, and is configured for controlling on and off of the negentropy separator; the power switch is configured to control connection and disconnection of the external power supply; and the power indicator is connected to the power switch, and is configured to display different power indication signals when the power switch is on or off.

In some embodiments, the negentropy separator is connected to the battery assembly through a negentropy bridge module; and the negentropy bridge module is connected to the negentropy switch.

In some embodiments, the negentropy separator comprises a negentropic voltage output end and a negentropic voltage detection end; and the negentropic voltage output end is connected to the negentropy bridge module, and the negentropic voltage detection end is connected to the negentropy digital voltmeter through a negentropic voltage detection line.

In some embodiments, the electrode plate assembly is connected to the negentropic voltage output end through a negentropy output connection line; and the electrode plate assembly comprises two electrodes.

In some embodiments, the number of the negentropy separator is two, and the two negentropy separators are both connected to the negentropy bridge module.

In some embodiments, the entropy reduction instrument further comprises a plug board;

wherein one end of the plug board is connected to the power supply interface, and the other end of the plug board is connected to the negentropy digital voltmeter, the oscilloscope, and the display.

In some embodiments, the negentropy separator comprises a connecting rod, a copper tank assembly, and a magnetic ring;

the copper tank assembly is sleeved on an outer periphery of the connecting rod; and

the magnetic ring is sleeved on an outer periphery of the copper tank.

In some embodiments, the copper tank assembly comprises a first copper tank, a second copper tank, and a third copper tank;

the second copper tank is sleeved on an outer periphery of the first copper tank;

the third copper tank is sleeved on an outer periphery of the second copper tank; and

the first copper tank, the second copper tank, and the third copper tank are all in a circular tubular shape.

Additional aspects and advantages of the present disclosure will be described below, some of which will become apparent from the following description or from the implementation of the present disclosure.

### BRIEF DESCRIPTION OF THE DRAWINGS

To illustrate the technical solutions in the embodiments of the present disclosure or in the prior art more clearly, the accompanying drawings that are required for the description of the embodiments or the prior art will be briefly described below. Obviously, other accompanying drawings can be obtained by one of ordinary skill in the art without paying for creative work based on these drawings.

FIG. 1 is a perspective view of an entropy reduction instrument according to an embodiment of the present disclosure;

FIG. 2 is a perspective view of the entropy reduction instrument according to another embodiment of the present disclosure;

FIG. 3 is a circuit diagram of the entropy reduction instrument according to an embodiment of the present disclosure;

FIG. 4 is a structural diagram of a negentropy separator according to an embodiment of the present disclosure;

FIG. 5 is a cross-sectional view of the negentropy separator according to an embodiment of the present disclosure; and

FIG. 6 is a longitudinal-sectional view of the negentropy separator according to an embodiment of the present disclosure.

In the drawings: 10, housing; 20, battery assembly; 30, negentropy separator; 31, negentropic voltage output end; 32, negentropic voltage detection end; 33, connecting rod; 34, copper tank assembly; 341, first copper tank; 342, second copper tank; 343, third copper tank; 35, magnetic ring; 36, magnetic ring support frame; 40, electrode plate assembly; 41, electrode; 50, power supply interface; 60, negentropy digital voltmeter; 70, oscilloscope; 80, display; 90, negentropy switch; 100, power switch; 110, power indicator; 120, negentropy bridge module; 130, negentropic voltage detection line; 140, negentropy output connection line; and 150, plug board.

### DETAILED DESCRIPTION OF EMBODIMENTS

Embodiments of the present disclosure will be described in detail below with reference to the drawings. In the drawings, the same or similar references indicate the same or similar components or components having the same or similar functions. The embodiments are merely illustrative of the technical solutions of the present disclosure, and should not be construed as limitations to the present disclosure.

The present disclosure provides an entropy reduction instrument, which can reorganize the micro-structure of human bodies in a holistic format at the particle level to achieve the original healthy state of the human bodies. Moreover, it can also activate pare cells of the human bodies, recover the damaged body cells, and change the disordered state of entropy increase, thereby ordering the entropy increase of the human body, delaying aging, and prolonging life.

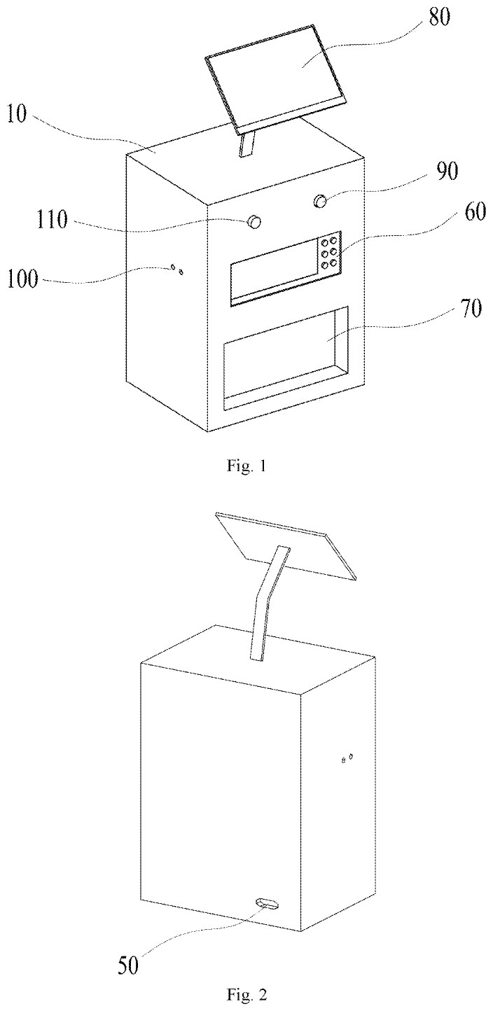

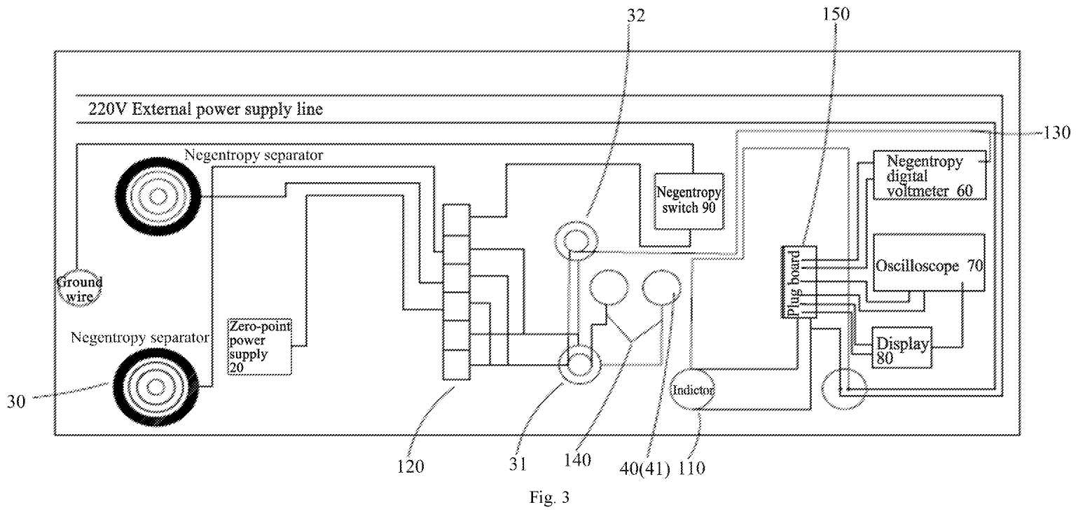

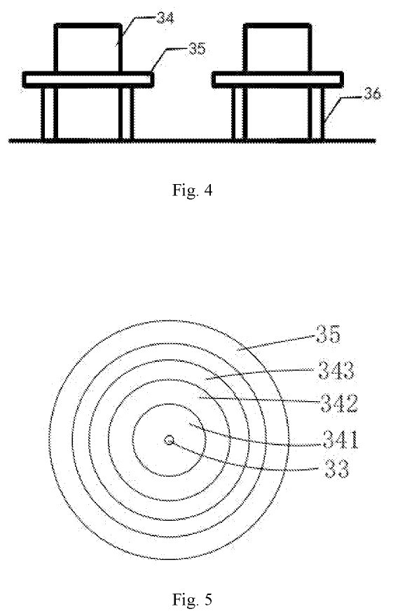

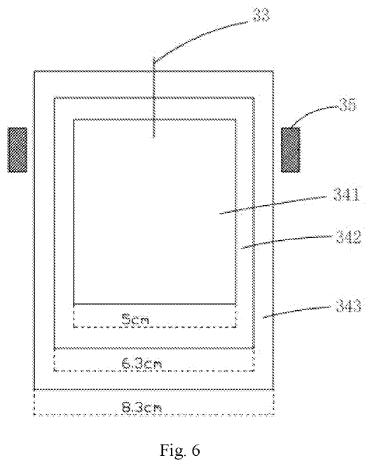

FIG. 1 is a perspective view of an entropy reduction instrument according to an embodiment of the present disclosure. FIG. 2 is a perspective view of the entropy reduction instrument according to another embodiment of the present disclosure. FIG. 3 is a circuit diagram of the entropy reduction instrument according to an embodiment of the present disclosure. FIG. 4 is a structural diagram of a negentropy separator according to an embodiment of the present disclosure. FIG. 5 is a cross-sectional view of the negentropy separator according to an embodiment of the present disclosure. FIG. 6 is a vertical-sectional view of the negentropy separator according to an embodiment of the present disclosure. Referring to FIGS. 1-6, this application provides an entropy reduction instrument, which includes a housing 10, a battery assembly 20, a negentropy separator 30, an electrode plate assembly 40, and a power supply interface 50.

The housing 10 constitutes an overall architecture of the entropy reduction instrument of the present disclosure. The housing 10 is hollow and irregular, and is provided with an accommodating cavity inside. The accommodating cavity is configured to hold various electronic components. In this embodiment, the housing 10 is made of acrylonitrile butadiene styrene (ABS) plastic, which provides good impact resistance, heat resistance, and low temperature resistance.

The battery assembly 20 is arranged in the accommodating cavity. A clamping part (such as protruding bars and bumps) are provided in an interior of the housing 10, so that the battery assembly 20 can be fixed in the clamping parts during assembly, thus avoiding loosening or shaking. The battery assembly 20 is configured to generate electrical energy, and the electrical energy contains negentropic energy.

The negentropy separator 30 is fixedly arranged in the accommodating cavity. For example, it can be fixed by a clamping part in the housing 10. The negentropy separator 30 is connected to the battery assembly 20, and is configured for receiving the electrical energy output from the battery assembly 20 and separating the negentropic energy from the electrical energy. Specifically, the battery assembly 20 outputs electrical energy containing negentropic energy. When the electrical energy enters the negentropy separator 30, the negentropic energy is separated from the electrical energy by the negentropy separator 30 to be applied to the human body. The negentropy separator 30 is configured to separate the negentropic energy, which has the greatest effect on the human body's reverse growth, from the electrical energy. Negentropic energy is the opposite to entropy increase, that is, entropy reduction. As the entropy increase is the process from life to death, the entropy reduction is the process to delay the entropy increase, so as to achieve inverse growth and a healthy and prolonged life.

The electrode plate assembly 40 is connected to the negentropy separator 30, and is configured for receiving and processing the negentropic energy separated by the negentropy separator 30 to generate uniform negentropic emission energy and apply it to the human body. Specifically, the negentropic energy is separated by the negative entropy separator 30 and then fed to the electrode plate assembly. The electrode plate assembly processes the negentropic energy to produce uniform negentropic emission energy that is beneficial to the human body. Then, the human body receives the uniform negentropic emission energy through the electrode plate assembly 40. In such cases, the spare cells of the human body can be activated, and the damaged body cells can be recovered, thus having a beneficial effect on the human body.

The power supply interface 50 is connected to the battery assembly 20 and the negentropy separator 30, and is configured to supply power to the battery assembly 20, the negentropy separator 30, and the electrode plate assembly 40 when being connected to an external power supply. Specifically, the power supply interface 50 is configured to connect to an external power supply, e.g., the commercial power supply (alternating current, 220V), so as to supply power to those electronic components.

The entropy reduction instrument provided herein can reorganize the micro-structure of human bodies in a holistic format at the particle level to achieve the original healthy state of the human bodies. Moreover, it can also activate pare cells of the human bodies, recover the damaged body cells, and change the disordered state of entropy increase, thereby ordering the entropy increase of the human body, delaying aging, and prolonging life.

In an embodiment, referring to FIGS. 1 and 3, the entropy reduction instrument provided herein further includes a negentropy digital voltmeter 60, an oscilloscope 70, and a display 80. The negentropy digital voltmeter 60 is connected to the negentropy separator 30, and is configured to detect the output voltage of the negentropy separator 30. In this case, the negentropy digital voltmeter 60 can detect and monitor the output voltage of the negentropy separator 30 in real time, in case the output voltage of the negentropy separator 30 is abnormal and thus cannot be monitored.

The oscilloscope 70 is connected to the negentropy separator 30, and is configured for monitoring physical parameters of the negentropy separator 30. Specifically, the oscilloscope 70 is configured to monitor the physical parameters of the negentropy separator 30 in real time, such as an output waveform state, an output voltage, and an output current.

The display 80 is connected to the oscilloscope 70, and is configured to synchronously transmit a display interface of the oscilloscope 70. Specifically, the display 80 is connected to the oscilloscope 70, so as to synchronously transmit the operating parameters and waveform states of the oscilloscope 70 in real time, facilitating the user to observe the operating state of the oscilloscope 70.

In an embodiment, referring to FIGS. 1 and 3, the entropy reduction provided herein further includes a negentropy switch 90, a power switch 100, and a power indicator 110. The negentropy switch 90 is connected to the negentropy separator 30, and is configured for controlling the on and off of the negentropy separator 30. Specifically, the negentropy switch 90 is configured to control connection and disconnection of the negentropy separator 30. For example, the negentropy separator 30 is on when the negentropy switch 90 is pressed, and the negentropy separator 30 is off when the negentropy switch 90 is pressed again.

The power switch 100 is configured to control the on and off of the external power supply. For example, when the power switch 100 is pressed or pushed to the on state, the external power supply supplies power to the negentropy separator 30, and when the power switch 100 is pressed again or pushed to the off state, the external power supply stops supplying power to the negentropy separator 30.

The power indicator 110 is connected to the power switch 100, and is configured to display different power indication signals when the power switch 100 is on or off. Specifically, for example, when the power switch 100 is on, the power indicator 110 is illuminated, and when the power switch 100 is off, the power indicator 110 is not illuminated.

In an embodiment, referring to FIG. 3, the negentropy separator 30 is connected to the battery assembly 20 through a negentropy bridge module 120. The negentropy bridge module 120 is also connected to the negentropy switch 90. Specifically, the negentropy separator 30 is connected to the battery assembly 20 and the negentropy switch 90, respectively.

In an embodiment, referring to FIG. 3, the negentropy separator 30 includes a negentropic voltage output end 31 and a negentropic voltage detection end 32. The negentropic voltage output end 31 is connected to the negentropy bridge module 120, and the negentropic voltage detection end 32 is connected to the negentropy digital voltmeter 60 through a negentropic voltage detection line 130. Specifically, the negentropic voltage output end 31 is connected to the negentropy bridge module 120 and the negentropic voltage detection end 32, and the negentropic voltage detection end 32 is connected to the negentropy digital voltmeter 60 through the negentropic voltage detection line 130. By these arrangements, the negentropy separator 30 can be monitored and detected in real time by the negentropy digital voltmeter 60.

In an embodiment, referring to FIG. 3, the electrode plate assembly 40 is connected to the negentropic voltage output end 31 through a negentropy output connection line 140. The electrode plate assembly 40 includes two electrodes 41. Specifically, the electrode plate assembly 40 is connected to the negentropic voltage output end 31 through the negentropy output connection line 140, thus achieving the connection between the electrode plate assembly 40 and the negentropy separator 30. The negentropic voltage output end 31 is connected to the negentropy bridge module 120, the negentropic voltage detection end 32, and the electrode plate assembly 40, respectively. The electrode plate assembly 40 includes two electrodes 41, one of which is positive and the other of which is negative. For example, when the health care and inverse growth are required, the positive electrode is placed at the Mingmen-acupoint, and the negative electrode is placed on Guanyuan-acupoint, Sanyinjiao-acupoint, or Yongquan-acupoint; when the heart-strengthening is required, the positive electrode is placed at Neiguan-acupoint or Shaohai-acupoint, and the negative electrode is place at the Sanyinjiao-acupoint. The entropy reduction instrument provided herein can be involved in the regulation of a wide range of chronic diseases, where the use duration can be chosen according to the individual's constitution, generally 40-60 minutes.

In an embodiment, referring to FIG. 3, the number of the negentropy separator 30 is two, and the two negentropy separators 30 are both connected to a negentropy bridge module 120. Specifically, the number of the negentropy separator 30 is two, and the two negentropy separators 30 are connected through the negentropy bridge module 120.

In an embodiment, referring to FIGS. 2 and 3, the entropy reduction instrument provided herein further includes a plug board 150. One end of the plug board 150 is connected to the power supply interface 50, and the other end of the plug board 150 is connected to the negentropy digital voltmeter 60, the oscilloscope 70, and the display 80 respectively. Specifically, the negentropy digital voltmeter 60, the oscilloscope 70, and the display 80 are connected to the power supply interface 50 through the plug board 150, thereby enabling the power supply of the external power supply to the negentropy digital voltmeter 60, the oscilloscope 70, and the display 80 through the power supply interface 50 and the plug board 150. In this embodiment, the negentropy digital voltmeter 60 is also connected to the plug board 150 through a power supply line of the negentropy digital voltmeter 60. The oscilloscope 70 is connected to the plug board 150 through a power supply line of the oscilloscope 70. The display 80 is connected to the plug board 150 via a power supply line of the display 80, and is connected to the oscilloscope 70 via a high definition (HD) data line.

In an embodiment, referring to FIGS. 4-6, the negentropy separator 30 includes a connecting rod 33, a copper tank assembly 34 sleeved on an outer periphery of the connecting rod 33, and a magnetic ring 35 sleeved on an outer periphery of the copper tank assembly 34. Specifically, the negentropy separator 30 includes the connecting rod 33 arranged at the center of the negentropy separator 30, the copper tank assembly 34 sleeved on the outer periphery of the connecting rod 33, and a magnetic ring 35 sleeved on the outer periphery of the copper tank assembly 34. In an embodiment, a bottom of the negentropy separator 30 is also provided with a magnetic ring support frame 36. By arranging the magnetic ring support frame 36 at the bottom of the negative entropy separator 30, the negentropy separator 30 can be fixedly arranged.

In an embodiment, referring to FIGS. 5-6, the copper tank assembly 34 includes a first copper tank 341 arranged internally, a second copper tank 342 sleeved on the outer periphery of the first copper tank 341, and a third copper tank 343 sleeved on the outer periphery of the second copper tank 342, where the first copper tank 341, the second copper tank 342, and the third copper tank 343 are all a circular tubular shape. Specifically, in this embodiment, the number of the copper tank assembly 34 is three, and all the three copper tanks 34 have a three-dimensional structure with a circular cross-section. The first copper tank 341 is located at the central position; the third copper tank 343 is located at the peripheral position; and the second copper tank 342 is located between the first copper tank 341 and the third copper tank 343. The first copper tank 341, the second copper tank 342, and the third copper tank 343 are all filled with water. The first copper tank 341 has an external diameter of 5 cm and a height of 8 cm, the second tank 342 has an external diameter of 6.3 cm and a height of 12.3 cm, the third tank 343 has an external diameter of 8.3 cm and a height of 15.5 cm. The first tank 341 is fixedly connected to the second tank 342 through a 0.6 cm diameter copper screw and a copper screw, and the second tank 342 is fixedly connected to the third tank 343 by a 0.6 cm diameter copper screw and a copper screw. By this arrangement, a fixed connection between the three tanks is achieved, thus avoiding loosening or shaking between the first tank 341, the second tank 342, and the third tank 343. The outer circumference of the third tank 343 is provided with a magnetic ring 35. The magnetic ring 35 is made of a ferrite universal magnetic material, and has an outer diameter of 20 cm, an inner diameter of 11 cm and a thickness of 2 cm.

The operational steps of the entropy reduction instrument provided by the present disclosure are described below.

(S1) A ground wire is connected.

(S2) A voltage measurement line of the negentropy digital voltmeter is connected.

(S3) A wave detection probe of the oscilloscope is connected.

(S4) The electrode plate assembly is connected.

(S5) The power supply line is connected.

(S6) The power supply indicator light is on.

(S7) The negentropy switch is turned on, where when the center button is pressed down, the negentropy switch is on; and when the center button recovers to the original state, the negentropy switch is off.

(S8) The display and the oscilloscope are turned on.

(S9) Whether the entropy reduction instrument can be operated normally is checked, which is described in detail below.

(1) Connection line items are checked, including:

whether the detection line of the negentropy digital voltmeter is connected; whether the electrode plate is connected; whether the waveform probe of the oscilloscope is connected; whether the ground line is connected; and whether the negentropy switch is turned on.

(2) The numerical status of the negentropy digital voltmeter and the waveform status of the oscilloscope are checked.

Under normal conditions, the negentropy digital voltmeter displays a range of values (−60-140±10); and when the negative entropy is applied to the human body, the negentropy digital voltmeter displays a range of values (0-15±10).

(3) When a fault occurs, the methods for checking and eliminating the fault are described below.

(i) The power indicator fails to light up, the negentropy digital voltmeter fails to display, and the display and oscilloscope cannot be turned on.

When encountering the above errors, it is to be first considered whether the power cable is connected, whether the external power switch is turned on, and whether a power failure exists in the room.

(ii) When turning on the negentropy digital voltmeter, the displayed value is not within the normal range, generally lower than the normal value.

When encountering the above failure, it is to be first considered whether the earth line is connected, since the earth line is the key to the normal operation of the entropy reduction instrument. If the failure is not solved, the following items are checked: whether the detection line of the negentropy digital voltmeter is connected correctly; and whether the digital display meter is operated by human error, resulting in wrong parameters. If the digital display meter is operated by human error, the auto key of the digital display meter is pressed to restore.

When the negentropy is applied to the body, the negentropy digital voltmeter shows no change in value. In this case, the following items are checked: whether the electrode plate is correctly connected to the entropy reduction instrument; whether the connection between the electrode plate and the connection cable of the electrode plate is loose; or whether an oxidation layer is formed on the surface of the electrode plate due to inappropriate cleaning after a long-term use.

As used herein, terms, such as “an embodiment”, “some embodiments”, “example”, “specific example”, or “some examples”, indicate that specific features, structures, materials or characteristics described with reference to the embodiment or example are included in at least one embodiment or example of the present disclosure. In this specification, the exemplary expressions of the above terms do not necessarily refer to the same embodiment or example. Furthermore, those specific features, structures, materials or characteristics may be combined in a suitable manner in any one or more of the embodiments or examples.

Obviously, the above embodiments are merely exemplary, and are not intended to limit the present disclosure. It should be understood that variations, modifications, and replacements made by those of ordinary skill in the art without departing from the spirit and scope of the present disclosure shall fall within the scope of the present disclosure defined by the appended claims.

What is claimed is:

1. An entropy reduction instrument, comprising:
a housing;
a battery assembly;
a negentropy separator;
an electrode plate assembly; and
a power supply interface;
wherein the housing is provided with an accommodating cavity inside;
the battery assembly is configured to generate electrical energy; and the electrical energy contains negentropic energy;
the negentropy separator is fixedly arranged in the accommodating cavity, and is connected to the battery assembly; and the negentropy separator is configured to receive the electrical energy output from the battery assembly and separate the negentropic energy from the electrical energy;
the electrode plate assembly is connected to the negentropy separator, and is configured for receiving and processing the negentropic energy separated by the negentropy separator to generate uniform negentropic emission energy and apply the uniform negentropic emission energy to human body; and
the power supply interface is connected to the battery assembly and the negentropy separator; and the power supply interface is configured to supply power to the battery assembly, the negentropy separator, and the electrode plate assembly after being connected to an external power supply.

2. The entropy reduction instrument of claim 1, further comprising:
a negentropy digital voltmeter;
an oscilloscope; and
a display;
wherein the negentropy digital voltmeter is connected to the negentropy separator, and is configured to detect an output voltage of the negentropy separator;
the oscilloscope is connected to the negentropy separator, and is configured for monitoring physical parameters and waveform state of the negentropy separator; and
the display is connected to the oscilloscope, and is configured to synchronously transmit a display interface of the oscilloscope.

3. The entropy reduction instrument of claim 1, further comprising:
a negentropy switch;
a power switch; and
a power indicator;
wherein the negentropy switch is connected to the negentropy separator, and is configured for controlling on and off of the negentropy separator;
the power switch is configured to control connection and disconnection of the external power supply; and
the power indicator is connected to the power switch, and is configured to display different power indication signals when the power switch is on or off.

4. The entropy reduction instrument of claim 3, wherein the negentropy separator is connected to the battery assembly through a negentropy bridge module; and the negentropy bridge module is connected to the negentropy switch.

5. The entropy reduction instrument of claim 4, wherein the negentropy separator comprises a negentropic voltage output end and a negentropic voltage detection end; and the negentropic voltage output end is connected to the negentropy bridge module, and the negentropic voltage detection end is connected to the negentropy digital voltmeter through a negentropic voltage detection line.

6. The entropy reduction instrument of claim 5, wherein the electrode plate assembly is connected to the negentropic voltage output end through a negentropy output connection line; and the electrode plate assembly comprises two electrodes.

7. The entropy reduction instrument of claim 4, wherein the number of the negentropy separator is two, and two negentropy separators are both connected to the negentropy bridge module.

8. The entropy reduction instrument of claim 2, further comprising:
a plug board;
wherein one end of the plug board is connected to the power supply interface, and the other end of the plug board is connected to the negentropy digital voltmeter, the oscilloscope, and the display.

9. The entropy reduction instrument of claim 1, wherein the negentropy separator comprises a connecting rod, a copper tank assembly, and a magnetic ring;
the copper tank assembly is sleeved on an outer periphery of the connecting rod; and
the magnetic ring is sleeved on an outer periphery of the copper tank.

10. The entropy reduction instrument of claim 9, wherein the copper tank assembly comprises a first copper tank, a second copper tank, and a third copper tank;
the second copper tank is sleeved on an outer periphery of the first copper tank;
the third copper tank is sleeved on an outer periphery of the second copper tank; and
the first copper tank, the second copper tank, and the third copper tank are all in a circular tubular shape.
